(19) United States
(12) Patent Application Publication
(10) Pub. No.: US 2011/0022616 A1
(43) Pub. Date: Jan. 27, 2011

(54) METHOD OF TREATING CYSTIC FIBROSIS USING OCEAN WATER

(76) Inventor: Holly Jeannine Hardin, O'Fallon, MO (US)

Correspondence Address: Daniel Boudwin, 111 Yorktown Rd, Woolwich, NJ, 08085, US

(21) Appl. No.: 12/843,780

(22) Filed: Jul. 26, 2010

Related U.S. Application Data
(60) Provisional application No. 61/271,828, filed on Jul. 27, 2009.

(51) Int. Cl. G06F 17/30; A61M 16/14

(52) U.S. Cl. 707/758; 128/203.12; 707/E17.041

(57) ABSTRACT

The present invention provides a method of treating respiratory disorders, such as cystic fibroses, through misting ocean water in a closed room where a patient resides.

### CROSS REFERENCE TO RELATED APPLICATION

This application claims the benefit of U.S. Provisional Application No.

61/271,828 filed on Jul. 27th, 2009, entitled “Sea Spray”.

### BACKGROUND OF THE INVENTION

1. Field of the Invention

The present invention relates to a method of treating cystic fibrosis and related respiratory and allergy disorders.

2. Description of the Prior Art

Cystic fibrosis is a respiratory disease that causes thick, sticky mucus to build up in the lungs and digestive tract of patients. Many children and young adults suffer from this chronic lung disease. Many of those with the disease are unable to perform daily activities, and some cases may result in death. Numerous methods of treating the disease have been proposed and are widely used, however, not all patients are responsive to the known types of treatments. Present treatments in the medical field include antibiotics to prevent and treat lung and sinus infections, DNAse enzyme replacement therapy used to thin mucus and make it easier to cough up, flu vaccines and pneumococcal polysaccharide vaccines provided on a yearly basis and, in extreme cases, a lung transplant may have to be done. Yet the most common treatments include inhaled medications and oxygen therapy. While substances such as saline have proved beneficial, results are not consistent on all patients, and symptoms may not be completely alleviated. Saline is commonly used in a mist, such as through a nebulizer, where the patient has to wear a device over their face. Other device such as croup tents isolate the patient in a small tent filled with a vapor or mist. The above two mentioned inhalant devices can be uncomfortable and can limit the patients mobility during treatment. The present invention attempts to alleviate these problems and provide an improved treatment method for patients suffering from respiratory diseases, as well as allergy problems.

### SUMMARY OF THE INVENTION

Recent monitoring of cystic fibrosis patients has shown a pattern of decreased symptoms when the patient is in or near the ocean. Symptoms have improved most dramatically in patients that surf regularly. The heightened benefit of ocean water, while surfing, leads to the assumption that the misted water is providing a direct benefit. While it is unknown exactly why ocean water causes such a benefit, the results are conclusive that it does. It is thought that the concentration of salt in the ocean water, or other bacteria, enzymes, and matter unique to ocean water, or at least unique in concentration, may be providing this benefit.

It is therefore an object of the present invention to provide commercially available and packaged ocean salt water for misting into a room where a patient resides.

Another object of the present invention is to gather results from the patients, compile the data based on origin of the ocean water, and isolate the beneficial matter in the ocean water.

Other objects, features and advantages of the present invention will become apparent from the following detailed description.

### DETAILED DESCRIPTION OF THE INVENTION

The present invention discloses a method for treating respiratory diseases, such as cystic fibrosis, as well as allergy related disorders, by using salt water taken directly from the ocean. Ocean water can be collected in large quantities and transported to a processing plant. At the plant the water can be tested for harmful bacteria. If harmful bacteria is found the water can be sterilized using common techniques such as chemicals or heat. If no harmful bacteria is found, or after the water has been sterilized, the water can be packaged into individual containers for commercial sale. These packaged products can then be shipped to pharmacies for the consumer, or patient, to purchase. The patient can bring the ocean water and a typical misting device or humidifier to a closed room and fill the device with the ocean water. A salt water mist will fill the room, in which the patient will sit in until symptoms improve.

Results relating to the treatment can be gathered by the patient's doctor and, upon patient consent, provided to the ocean water collection company. The data can be compiled into a database where its results can be sorted by water origin. Comparing success rates of water from different regions can help narrow down the helpful elements, bacteria, or other matter in the ocean water that prove so helpful to suffering patients. With the beneficial matter isolated, future advance can be made towards the treatment of these chronic respiratory diseases.

With respect to the above description then, it is to be realized that the optimum method or compositions to include variations deemed readily apparent and obvious to one skilled in the art, and all equivalent relationships to those described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact method and composition shown and described, and accordingly, all suitable modifications and equivalents may be resorted to, falling within the scope of the invention.

I claim:

1. A method of treating respiratory diseases using ocean water, comprising the steps of:
collecting salt water from the ocean, transporting said salt water to a processing plant, packaging said salt water in commercially marketable containers, distributing said packaged water to patients, and misting the salt water into a closed room where the patient is located.

2. The method of claim 1, further comprising the step of testing said salt water for harmful bacteria prior to packaging.

3. The method of claim 2, further comprising the step of sterilizing said salt water if harmful bacteria are found.

4. The method of claim 1, further comprising the step of compiling a database of success rates amongst users, comparing said rates by region of water origin and testing said water at said origin location for unique beneficial matter and concentrations thereof.
